SEQUENCE LISTING (7 sequences)

SEQ ID NO: 1 - DNA, 1831 bp, Rattus norvegicus
gccgctgcacagccccggcttcccgcgggggcggccatggtccggcgcttggcccggggctgctggtccgcgttctgggactacgagacgcctaaggtgatcgtggtgcggaatcggcgcctgggattcgtgcaccgcatggtgcagcttctcatcctgctttacttcgtgtggtacgtcttcatcgtgcagaaaagctaccaggacagcgagaccggaccggagagctccatcatcaccaaagtcaaggggatcaccatgtcggaagacaaagtgtgggacgtggaggaatacgtaaagcccccggaggggggcagtgtagtcagcatcatcaccaggatcgaggttaccccttcccagaccttgggaacatgcccagagagcatgagggttcacagctctacctgccattcagacgacgactgtattgccggacagctggacatgcaaggcaatgggattcgcacagggcactgtgtaccctattaccatggggactccaagacctgcgaggtgtcagcctggtgcccggtggaggatggaacttctgacaaccattttctgggtaaaatggccccaaatttcaccatcctcatcaagaacagcatccactaccccaagttcaagttctcaaagggcaacattgcaagccagaagagtgactacctcaagcattgcacatttgatcaggactctgacccatactgtcccatcttcaggctgggtttcattgttgagaaggcaggagagaacttcacagaactggcacacaagggcggtgtcattggagtcatcatcaactggaactgtgacctggacttgtctgaatcagagtgcaaccccaaatattctttccggaggctcgaccccaagtatgaccctgcctcctcaggctacaacttcaggtttgccaagtattacaagataaacggcactaccaccactcgaactctcatcaaagcctatgggattcgaatcgatgttatcgtgcatggacaggcagggaaattcagtctcattcccaccatcatcaatctggccactgctctgacctccatcggggtgggctccttcctgtgtgactggattttgttaacgttcatgaacaaaaacaagctctacagccataagaagttcgacaaggtgcgtactccaaagcatccctcaagtagatggcctgtgacccttgcccttgtcttgggccagatccctcccccacctagtcactactcccaggatcagccacccagccctccatcaggtgaaggaccaactttgggagaaggggcagagctaccactggctgtccagtctcctcggccttgctccatctctgctctgactgagcaggtggtggacacacttggccagcatatgggacaaagacctcctgtccctgagccttcccaacaggactccacatccacggaccccaaaggtttggcccaactttgatctcatcctcactaaactacagacctggacctgggaaggcagagacagctttggctgctaaggcagtcctagagaagatctgcgctcttcagtaaccatgtccatgtgactgggaaacagaaacctgtgcaagaggacaggcgtcttgctttagcccaagcttacattcttcctctccctaaggcctctggggagaagtgggttccctgccatctcctttcccaacagaactcctcataggacctttccctgctcacctcttgtactctcatacagtattcagggaccccaagttaggggctatgctcctgttgtataatttcaagcccccctttagaagttgcagcatgctgagttcaataaaccagtgatgagc

SEQ ID NO: 2 - DNA, 1344 bp, Homo sapiens
atggccgccgcccagcccaagtaccccgccggggcgaccgcccggcgcctggcccggggctgctggtccgccctctgggactacgagacgcccaaggtgatcgtggtgaggaaccggcgcctgggggtcctgtaccgcgccgtgcagctgctcatcctgctctacttcgtgtggtacgtattcatcgtgcagaaaagctaccaggagagcgagacgggccccgagagctccatcatcaccaaggtcaaggggatcaccacgtccgagcacaaagtgtgggacgtggaggagtacgtgaagccccccgagagcataagggtccacaacgccacctgcctctccgacgccgactgcgtggctggggagctggacatgctgggaaacggcctgaggactgggcgctgtgtgccctattaccaggggccctccaagacctgcgaggtgttcggctggtgcccggtggaagatggggcctctgtcagccaatttctgggtacgatggccccaaatttcaccatcctcatcaagaacagcatccactaccccaaattccacttctccaagggcaacatcgccgaccgcacagacgggtacctgaagcgctgcacgttccacgaggcctccgacctctactgccccatcttcaagctgggctttatcgtggagaaggctggggagagcttcacagagctcgcacacaagggtggtgtcatcggggtcattatcaactgggactgtgacctggacctgcctgcatcggagtgcaaccccaagtactccttccggaggcttgaccccaagcacgtgcctgcctcgtcaggctacaacttcaggtttgccaaatactacaagatcaatggcaccaccacccgcacgctcatcaaggcctacgggatccgcattgacgtcattgtgcatggacaggccgggaagttcagcctgattcccaccattattaatctggccacagctctgacttccgtcggggtgggctccttcctgtgcgactggatcttgctaacattcatgaacaaaaacaaggtctacagccataagaaatttgacaaggtgtgtacgccgagccacccctcaggtagctggcctgtgacccttgcccgtgtattgggccaggcccctcccgaacccggccaccgctccgaggaccagcaccccagccctccatcaggccaggagggccaacaaggggcagagtgtggcccagccttcccgcccctgcggccttgccccatctctgccccttctgagcagatggtggacactcctgcctccgagcctgcccaagcctccacacccacagaccccaaaggtttggctcaactctga

SEQ ID NO: 3 - DNA, 22 bp, Artificial Sequence
  Oligonucleotide antisense
gtagtggatgctgttcttgatg

SEQ ID NO: 4 - DNA, 22 bp, Artificial Sequence
  Oligonucleotide antisense
gtagttgaggctcttgttgatg

SEQ ID NO: 5 - DNA, 20 bp, Artificial Sequence
  Oligonucleotide antisense
accaggatcgaggttacccc

SEQ ID NO: 6 - DNA, 21 bp, Artificial Sequence
  Oligonucleotide antisense
gagctgtgaaccctcatgctc

SEQ ID NO: 7 - DNA, 23 bp, Artificial Sequence
  Oligonucleotide antisense
tcccagaccttgggaacatgccc

(19) United States
(12) Patent Application Publication
(10) Pub. No.: US 2008/0119423 A1
(43) Pub. Date: May 22, 2008

(54) Methods and Materials For Modulating p2x2

(76) Inventors: Samuel J. Shuster, Edina, MN (US); Ulf N.G. Arvidsson, Lidingo (SE); Laura S. Stone, St. Louis Park, MN (US); Hong-Yan Zhang, Sundbyberg (SE); Lucy Vulchanova Hart, St. Paul, MN (US)

Correspondence Address: FISH & RICHARDSON P.C., PO BOX 1022, MINNEAPOLIS, MN, 55440-1022, US

(21) Appl. No.: 10/500,449

(22) PCT Filed: Dec. 31, 2002
(86) PCT No.: PCT/US02/41833

§ 371 Date: Dec. 3, 2004

Related U.S. Application Data
(60) Provisional application No. 60/346,155, filed on Dec. 31, 2001.

(51) Int. Cl. A61K 31/711; C07H 21/04; A61P 43/00; C12N 5/06

(52) U.S. Cl. 514/44; 536/24.5; 435/325; 435/375

(57) ABSTRACT

The invention relates to antisense oligonucleotides, compositions and methods useful for modulating the expression of P2X2. The compositions comprise antisense oligonucleotides targeted to nucleic acids encoding P2X2.

### TECHNICAL FIELD

This invention relates to antisense oligonucleotides targeted to specific nucleotide sequences. In particular, the invention pertains to antisense oligonucleotides targeted to the nucleic acid encoding the P2X2 purinoreceptor, and to their use for reducing cellular levels of P2X2.

### BACKGROUND

The P2X2 receptor subunit belongs to a family of ATP gated ion channels and was originally isolated from PC12 cells. See, Brake et al., 1994, Nature, 371:519-523. P2X2 is one of seven known subunits that belong to this family (P2X1-7). Each subunit appears to have two transmembrane domains, a large extracellular loop, and intracellular N- and C-termini. See, Surprenant et al., 1995, Trends in Neurosci., 18:224-229. P2X receptor subunits are present in the central and peripheral nervous system as well as on non-neuronal cells, and each has a distinct expression pattern.

In rodents, P2X2 is mainly expressed in the nervous system. See, Collo et al., 1996, J. Neurosci., 16:2495-2507; Vulchanova et al., 1996, Proc. Natl. Acad. Sci. USA, 93:8063-8067; and Vulchanova et al., 1997, Neuropharmacology, 36:1229-1242. P2X2 is present in rat dorsal root ganglia and spinal cord. In dorsal root ganglia, P2X2 is localized mainly in small neurons, most of which are thought to sense painful stimuli. In spinal cord, P2X2 is present in the central terminal of sensory neurons in superficial dorsal horn, and in intrinsic spinal cord neurons. P2X2 is similarly localized in non-human primates.

The presence of P2X2 in superficial dorsal horn of spinal cord and in sensory neurons suggests that P2X2 has a role in pain signaling. Further suggesting a role of P2X2 in pain signaling is the apparent association of P2X2 with the P2X3 receptor, which is strongly implicated in pain sensation.

### SUMMARY

Antisense oligonucleotides can be targeted to specific nucleic acid molecules, and to thereby reduce expression of specific nucleic acid molecules. For example, antisense oligonucleotides targeted to P2X2 mRNA could be used therapeutically to reduce the level of P2X2 receptors in a patient suffering from chronic pain.

One challenge in generating useful antisense oligonucleotides is identifying nucleic acid segments within a target mRNA that are suitable targets for antisense molecules. Antisense oligonucleotides typically are targeted to segments within a target mRNA based on, for example, the function of those segments (e.g., translation start site, coding sequence, etc.). Such targeting approaches are often unsuccessful because they do not account for the tertiary structure of the specific mRNA target. Native mRNA generally is folded into a complex secondary and tertiary structure, rendering sequences on the interior of such folded molecules inaccessible to antisense oligonucleotides. Only antisense molecules directed to accessible portions of a native mRNA could effectively hybridize to the mRNA and potentially bring about a desired result. Therefore, P2X2 antisense molecules useful to reduce levels of P2X2 and alleviate pain should be targeted to accessible mRNA sequences.

The invention provides isolated antisense oligonucleotides that specifically hybridize to accessible regions of native P2X2 mRNA. Such antisense oligonucleotides can inhibit production of P2X2 and can be used therapeutically to reduce P2X2 levels. The invention provides isolated antisense oligonucleotides that specifically hybridize within an accessible region of P2X2 mRNA in its native form, wherein the antisense oligonucleotides inhibit production of P2X2. The invention also provides methods for decreasing production of P2X2 in cells or tissues. The method involves contacting cells or tissues with an antisense oligonucleotide that specifically hybridizes within an accessible region of P2X2 mRNA.

The invention features isolated antisense oligonucleotides consisting essentially of 10 to 50 nucleotides and compositions containing such antisense oligonucleotides. The oligonucleotide can specifically hybridize within an accessible region of the rat P2X2 mRNA in its native state, wherein the accessible region is defined by nucleotides 231 through 249, 589 through 617, 650 through 668, 829 through 846, 940 through 957, 1246 through 1273, or 1429 through 1446. The antisense oligonucleotides of the invention also can inhibit the production of P2X2.

The invention also features compositions comprising such isolated antisense oligonucleotides. The compositions can include a plurality of isolated antisense oligonucleotides, wherein each antisense oligonucleotides base specifically hybridizes within a different accessible region.

The invention also features a nucleic acid construct that includes a regulatory element operably linked to a nucleic acid encoding a transcript that specifically hybridizes within one or more accessible regions of P2X2 mRNA in its native form. Host cell that contain such nucleic acids are also provided.

Unless otherwise defined, all technical and scientific terms used herein have the same meaning as commonly understood by one of ordinary skill in the art to which this invention belongs. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is the nucleotide sequence of rat P2X2 (SEQ ID NO:1). GenBank Accession No. U14414.

FIG. 2 is the nucleotide sequence of human P2X2 (SEQ ID NO:2). GenBank Accession No. NM016318.

FIG. 3A and FIG. 3B are line graphs depicting results of nociceptive testing in rats: 1) after catheterization but before induction of chronic neuropathic pain; 2) after induction of chronic neuropathic pain but before antisense treatment; and 3) after antisense treatment. FIG. 3A depicts results in rats subjected to a thermal stimulus, and FIG. 3B depicts results in rats subjected to a mechanical stimulus.

FIG. 4A and FIG. 4B are line graphs depicting the results of nociceptive testing in rats: 1) after catheterization but before induction of chronic inflammatory pain; 2) after induction of chronic inflammatory pain but before antisense treatment; and 3) after antisense treatment. FIG. 4A depicts results in rats subjected to a thermal stimulus, and FIG. 4B depicts results in rats subjected to a mechanical stimulus.

FIG. 5 is a bar graph depicting the relative P2X2 mRNA expression level in rat sensory neurons (L4 dorsal root ganglion) after induction of chronic inflammatory pain and after mismatch and antisense treatment.

### DETAILED DESCRIPTION

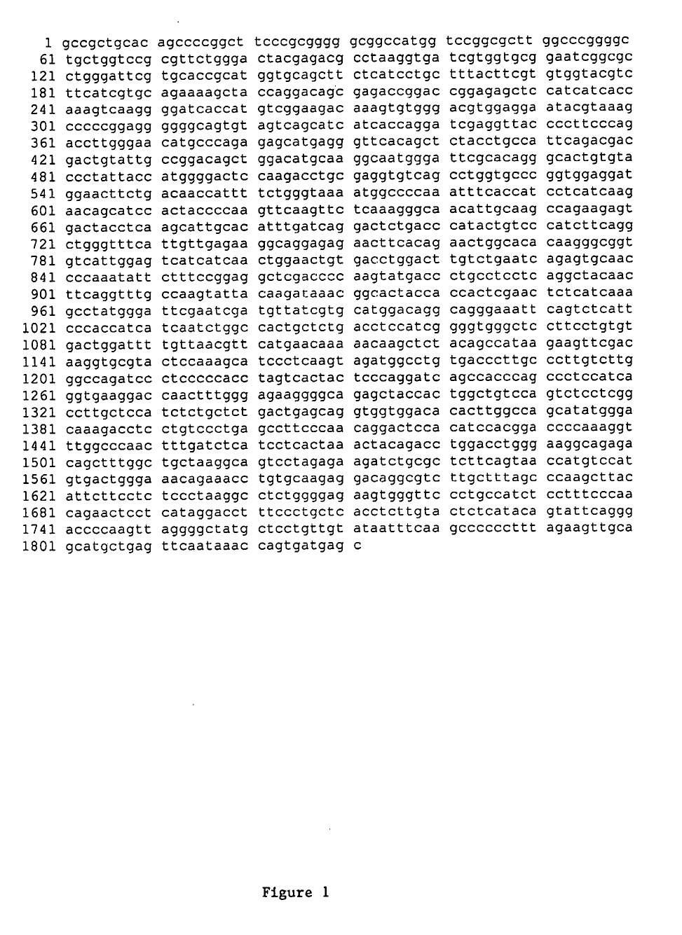

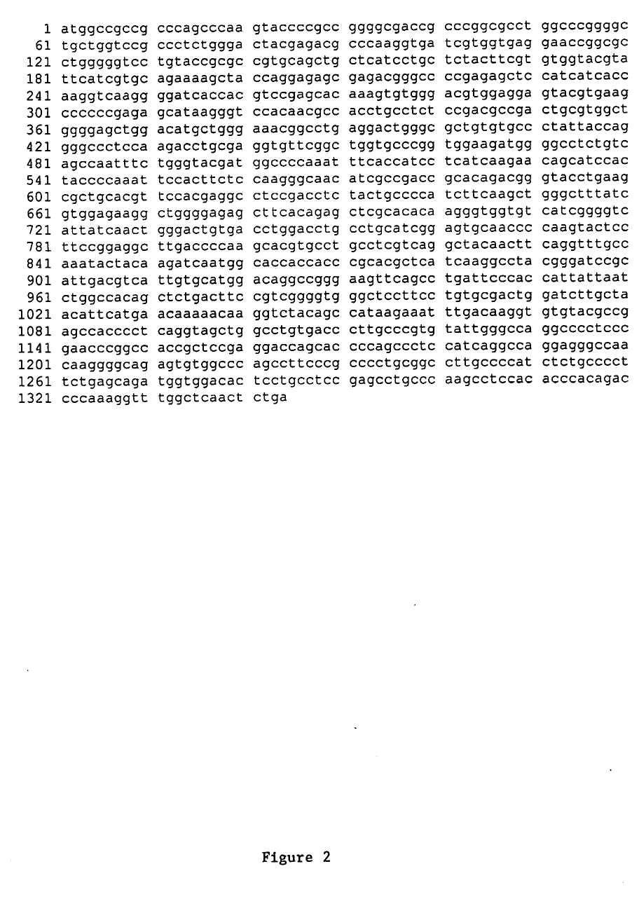

The invention provides antisense molecules, particularly oligonucleotides, useful for modulating the function of target nucleic acid molecules. A “target nucleic acid” can be RNA and can be DNA, including cDNA, genomic DNA, and synthetic (e.g., chemically synthesized) DNA. A target nucleic acid can be double-stranded, and can be single-stranded (i.e., a sense or an antisense single strand). In some embodiments, a target nucleic acid encodes a P2X2 polypeptide. Thus, “target nucleic acids” include DNA encoding P2X2, RNA (including pre-mRNA and mRNA) transcribed from such DNA, and cDNA derived from such RNA. FIGS. 1 and 2 provide nucleic acid sequences encoding rat and human P2X2 polypeptides (SEQ ID NO:1 and SEQ ID NO:2, respectively). An “antisense” molecule contains nucleic acids or nucleic acid analogs, and can specifically hybridize to a target nucleic acid. “Antisense technology” refers to the modulation of function of a target nucleic acid by an antisense oligonucleotide.

“Hybridization” means hydrogen bonding, which can be Watson-Crick, Hoogsteen, or reversed Hoogsteen hydrogen bonding, between complementary nucleoside or nucleotide bases. “Complementary” refers to the capacity for precise pairing between two nucleotides. For example, adenine and thymine, and guanine and cytosine, respectively, are complementary nucleotide bases (often referred to as “bases”) that pair via hydrogen bonds.

If a nucleotide at a particular position of a target nucleic acid is capable of hydrogen bonding with a nucleotide within an oligonucleotide (e.g., a candidate antisense molecule), then the oligonucleotide is considered to be complementary to the target nucleic acid at that position. An oligonucleotide and a target nucleic acid are complementary to each other when a sufficient number of corresponding positions in each molecule are occupied by nucleotides that can hydrogen bond with each other. Thus, “specifically hybridizable” refers to such degree of complementarity or precise pairing that stable and specific binding occurs between an oligonucleotide and a target nucleic acid.

It is understood in the art that the sequence of an antisense oligonucleotide need not be 100% complementary to that of its target nucleic acid to be specifically hybridizable. An antisense oligonucleotide is specifically hybridizable when (a) binding of the oligonucleotide to the target nucleic acid interferes with the normal function of the target DNA or RNA, and (b) there is sufficient complementarity to avoid non-specific binding of the antisense oligonucleotide to non-target nucleic acids when specific binding is desired, i.e., under in vitro assay conditions or under in vivo physiological conditions for assays or therapy.

The stringency of in vitro hybridization conditions can be adjusted to affect the degree of complementarity or precise pairing required for specific hybridization of an oligonucleotide to a target nucleic acid. The stringency of in vitro hybridization depends on temperature, time, and salt concentration (see, e.g., Sambrook et al., Molecular Cloning: A Laboratory Manual, Cold Spring Harbor Laboratory Press, NY, 1989). Typically, conditions of high to moderate stringency are used for specific hybridization in vitro, such that hybridization occurs between substantially similar nucleic acids, but not between dissimilar nucleic acids. Specific hybridization conditions are hybridization in 5×SSC (0.75 M sodium chloride/0.075 M sodium citrate) for 1 hour at 40° C. with shaking, followed by washing 10 times in 1×SSC at 40° C. and 5 times in 1×SSC at room temperature. Oligonucleotides that specifically hybridize to a target nucleic acid can be identified by recovering the oligonucleotides from oligonucleotide/target hybridization duplexes (e.g., by boiling) and sequencing the recovered oligonucleotides.

In vivo hybridization conditions are intracellular conditions (e.g., physiological pH and intracellular ionic conditions) that affect the hybridization of antisense oligonucleotides to target sequences. In vivo conditions can be mimicked in vitro using relatively low stringency conditions, such as those used in the RiboTAG™ technology described below. For example, hybridization can be carried out in vitro in 2×SSC (0.3 M sodium chloride/0.03 M sodium citrate), 0.1% SDS at 37° C. Alternatively, a wash solution containing 4×SSC, 0.1% SDS can be used at 37° C., with a final wash in 1×SSC at 45° C.

Specific hybridization of an antisense molecule with a target nucleic acid can interfere with the normal function of the target nucleic acid. For a target DNA, antisense technology can disrupt replication and transcription. For a target RNA, antisense technology can disrupt, for example, translocation of the RNA to the site of protein translation, translation of protein from the RNA, splicing of the RNA to yield one or more mRNA species, and catalytic activity of the RNA. Antisense technology can also facilitate nucleolytic degradation of a target RNA. The overall effect of such interference with target nucleic acid function is, in the case of a nucleic acid encoding P2X2, modulation of the expression of P2X2. In the context of the present invention, “modulation” means a decrease in the expression of a gene and/or a decrease in cellular levels or activity of the protein encoded by a gene.

### Identification of Target Sequences for P2X2 Antisense Oligonucleotides

Antisense oligonucleotides preferably are directed at specific regions within a target nucleic acid. The process of “targeting” an antisense oligonucleotide typically begins with identifying a candidate target nucleic acid whose function is to be modulated. This nucleic acid can be, for example, a cellular gene (or mRNA transcribed from the gene) whose expression is associated with a particular disorder or disease state.

The targeting process also involves identifying a region or regions within a target nucleic acid where an antisense interaction can occur such that a desired effect is achieved. The desired effect can be, for example, modulation of P2X2 expression or detection of P2X2 mRNA (e.g., by using a detectably labeled antisense oligonucleotide). Antisense oligonucleotides have been directed at regions encompassing the translation initiation or termination codon of the open reading frame (ORF) of a gene. Antisense oligonucleotides have also been directed at ORFs, at the 5′ and 3′ untranslated regions of genes, and at intron regions and intron-exon junction regions.

Knowledge of the sequence and domain structure (e.g., the location of translation initiation codons, exons, or introns) of a target nucleic acid, however, is generally not sufficient to ensure that an antisense oligonucleotide directed to a specific region will effectively bind to and modulate the function of the target nucleic acid. In its native state, an mRNA molecule is folded into complex secondary and tertiary structures, and sequences on the interior of such folded structures generally are inaccessible to antisense oligonucleotides. For maximal effectiveness, antisense oligonucleotides can be directed to regions of a target mRNA that are most accessible, i.e., regions at or near the surface of a folded mRNA molecule.

Accessible regions of an mRNA molecule can be identified by, for example, the RiboTAG™ method, or mRNA Accessible Site Tagging (MAST), as described in PCT App. No. SE01/02054.

Using the RiboTAG™ method, oligonucleotides that can interact with a test mRNA in its native state (i.e., under physiological conditions) are selected and sequenced, thus leading to the identification of regions within the test mRNA that are accessible to antisense molecules. In a version of the RiboTAG™ protocol, the test mRNA is produced by in vitro transcription and is then immobilized, for example by covalent or non-covalent attachment to a bead or a surface (e.g., a magnetic bead). The immobilized test mRNA is then contacted by a population of oligonucleotides, wherein a portion of each oligonucleotide contains a different, random region. Oligonucleotides that can hybridize to the test mRNA under conditions of low stringency are separated from the remainder of the population (e.g., by precipitation of the magnetic beads). The selected oligonucleotides then can be amplified and sequenced; these steps of the protocol are facilitated if the random regions within each oligonucleotide are flanked on one or both sides by non-random regions that can serve as primer binding sites for PCR amplification.

In general, oligonucleotides useful for RiboTAG™ technology contain between 15 and 18 random bases, flanked on either side by non-random regions. These oligonucleotides are contacted by a test mRNA under conditions that do not disrupt the native structure of the mRNA (e.g., in the presence of medium pH buffering, salts that modulate annealing, and detergents and/or carrier molecules that minimize non-specific interactions). Typically, hybridization is carried out at 37 to 40° C., in a solution containing × to 5×SSC and about 0.1% SDS. Non-specific interactions can be further minimized by blocking the non-random sequence(s) in each oligonucleotide with the primers that will be used for PCR amplification of the selected oligonucleotides.

As described herein, accessible regions of nucleic acids encoding rat P2X2 have been mapped. Thus, antisense oligonucleotides of the invention can specifically hybridize within one or more accessible regions defined by: nucleotides 231 through 249, 589 through 617, 650 through 668, 829 through 846, 940 through 957, 1246 through 1273, or 1429 through 1446 of SEQ ID NO:1. Using the methods disclosed herein, those of skill in the art can, as a matter of routine experimentation, identify accessible regions of nucleic acids encoding human P2X2 (SEQ ID NO:2).

Once accessible regions of a target nucleic acid have been identified, those of skill in the art can, as a matter of routine, design antisense oligonucleotides that specifically hybridize to the target nucleic acid. It should be noted that an antisense oligonucleotide may consist essentially of a nucleotide sequence that specifically hybridizes with an accessible region set out above. Such antisense oligonucleotides, however, may contain additional flanking sequences of 5 to 10 nucleotides at either end. Flanking sequences can include, for example, additional sequence of the target nucleic acid or primer sequence.

For maximal effectiveness, further criteria can be applied to the design of antisense oligonucleotides. Such criteria are known in the art, and are widely used, for example, in the design of oligonucleotide primers. These criteria include the lack of predicted secondary structure of a potential antisense oligonucleotide, an appropriate GC content (e.g., approximately 50%), and the absence of sequence motifs such as single nucleotide repeats (e.g., GGGG runs).

### P2X2 Antisense Oligonucleotides

Once one or more accessible target regions have been identified, antisense oligonucleotides sufficiently complementary to the target nucleic acid (i.e., that hybridize with sufficient strength and specificity to give the desired effect) can be synthesized. In the context of the present invention, the desired effect is the modulation of P2X2 expression such that cellular P2X2 levels are reduced. The effectiveness of an antisense oligonucleotide to modulate expression of a target nucleic acid can be evaluated by measuring levels of the mRNA or protein products of the target nucleic acid (e.g., by Northern blotting, RT-PCR, Western blotting, ELISA, or immunohistochemical staining).

In some embodiments, it may be useful to target multiple accessible regions of a target nucleic acid. In such embodiments, multiple antisense oligonucleotides can be used that each specifically hybridize to the same accessible region or to different accessible regions. Multiple antisense oligonucleotides can be used together or sequentially.

The antisense oligonucleotides in accordance with this invention preferably are from about 10 to about 50 nucleotides in length (e.g., 12 to 40, 14 to 30, or 15 to 25 nucleotides in length). Antisense oligonucleotides that are 15 to 23 nucleotides in length are particularly useful. However, an antisense oligonucleotide containing even fewer than 10 nucleotides (for example, a portion of one of the preferred antisense oligonucleotides) is understood to be included within the present invention so long as it demonstrates the desired activity of inhibiting expression of the P2X2 purinoreceptor.

An “antisense oligonucleotide” can be an oligonucleotide as described herein. The term “oligonucleotide” refers to an oligomer or polymer of ribonucleic acid (RNA) or deoxyribonucleic acid (DNA) or analogs thereof. This term includes oligonucleotides composed of naturally occurring nucleotide bases, sugars and covalent internucleoside (backbone) linkages, as well as oligonucleotides having non-naturally occurring portions which function similarly. Such modified or substituted oligonucleotides are often preferred over native forms because of desirable properties such as, for example, enhanced cellular uptake, enhanced affinity for a nucleic acid target, and increased stability in the presence of nucleases.

While antisense oligonucleotides are a preferred form of antisense molecules, the present invention includes other oligomeric antisense molecules, including but not limited to oligonucleotide analogs such as those described below. As is known in the art, a nucleoside is a base-sugar combination, wherein the base portion is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric molecule. The respective ends of this linear polymeric structure can be further joined to form a circular structure, although linear structures are generally preferred. Within the oligonucleotide structure, the phosphate groups are commonly referred to as forming the internucleoside backbone of the oligonucleotide. The normal linkage or backbone of RNA and DNA is a 3′ to 5′ phosphodiester linkage.

P2X2 antisense oligonucleotides that are useful in the present invention include oligonucleotides containing modified backbones or non-natural internucleoside linkages. As defined herein, oligonucleotides having modified backbones include those that have a phosphorus atom in the backbone and those that do not have a phosphorus atom in the backbone. For the purposes of this specification, and as sometimes referenced in the art, modified oligonucleotides that do not have a phosphorus atom in their internucleoside backbone also can be considered to be oligonucleotides.

Modified oligonucleotide backbones can include, for example, phosphorothioates, chiral phosphorothioates, phosphorodithioates, phosphotriesters, aminoalkylphosphotriesters, methyl and other alkyl phosphonates (e.g., 3′-alkylene phosphonates and chiral phosphonates), phosphinates, phosphoramidates (e.g., 3′-amino phosphoramidate and aminoalkylphosphoramidates), thionophosphoramidates, thionoalkylphosphonates, thionoalkylphosphotriesters, and boranophosphates having normal 3′-5′ linkages, as well as 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included. References that teach the preparation of such modified backbone oligonucleotides are provided, for example, in U.S. Pat. Nos. 4,469,863 and 5,750,666.

P2X2 antisense molecules with modified oligonucleotide backbones that do not include a phosphorus atom therein can have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. References that teach the preparation of such modified backbone oligonucleotides are provided, for example, in U.S. Pat. Nos. 5,235,033 and 5,596,086.

In another embodiment, a P2X2 antisense molecule can be an oligonucleotide analog, in which both the sugar and the internucleoside linkage (i.e., the backbone) of the nucleotide units are replaced with novel groups, while the base units are maintained for hybridization with an appropriate nucleic acid target. One such oligonucleotide analog that has been shown to have excellent hybridization properties is referred to as a peptide nucleic acid (PNA). In PNA molecules, the sugar-backbone of an oligonucleotide is replaced with an amide containing backbone (e.g., an aminoethylglycine backbone). The nucleotide bases are retained and are bound directly or indirectly to aza nitrogen atoms of the amide portion of the backbone. References that teach the preparation of such modified backbone oligonucleotides are provided, for example, in Nielsen et al., 1991, Science, 254:1497-1500, and in U.S. Pat. No. 5,539,082.

Other useful P2X2 antisense oligonucleotides can have phosphorothioate backbones and oligonucleosides with heteroatom backbones, and in particular CH₂NHOCH₂, CH₂N(CH₃)OCH₂, CH₂ON(CH₃)CH₂, CH₂N(CH₃)N(CH₃)CH₂, and ON(CH₃)CH₂CH₂ (wherein the native phosphodiester backbone is represented as OPOCH₂) as taught in U.S. Pat. No. 5,489,677, and the amide backbones disclosed in U.S. Pat. No. 5,602,240.

Substituted sugar moieties also can be included in modified oligonucleotides. P2X2 antisense oligonucleotides of the invention can comprise one or more of the following at the 2′ position: OH; F; O-, S-, or N-alkyl; O-, S-, or N-alkenyl; O-, S-, or N-alkynyl; or O-alkyl-O-alkyl, wherein the alkyl, alkenyl and alkynyl can be substituted or unsubstituted C₁ to C₁₀ alkyl or C₂ to C₁₀ alkenyl and alkynyl. Useful modifications also can include O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(C₂)ₙCH₃)]₂, where n and m are from 1 to about 10. In addition, oligonucleotides can comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, groups for improving the pharmacokinetic or pharmacodynamic properties of an oligonucleotide, and other substituents having similar properties. Other useful modifications include an alkoxyalkoxy group, e.g., 2′-methoxyethoxy (2′-OCH₂CH₂OCH₃), a dimethylaminooxyethoxy group (2′-O(CH₂)₂ON(CH₃)₂), or a dimethylamino-ethoxyethoxy group (2′-OCH₂OCH₂N(CH₂)₂). Other modifications can include 2′-methoxy (2′-OCH₃), 2′-aminopropoxy (2′-OCH₂CH₂CH₂NH₂), or 2′-fluoro (2′-F). Similar modifications also can be made at other positions within the oligonucleotide, such as the 3′ position of the sugar on the 3′ terminal nucleotide or in 2′-5′ linked oligonucleotides, and the 5′ position of the 5′ terminal nucleotide. Oligonucleotides also can have sugar mimetics such as cyclobutyl moieties in place of the pentofuranosyl group. References that teach the preparation of such substituted sugar moieties include U.S. Pat. Nos. 4,981,957 and 5,359,044.

Useful P2X2 antisense oligonucleotides also can include nucleotide base modifications or substitutions. As used herein, “unmodified” or “natural” nucleotide bases include the purine bases adenine (A) and guanine (G), and the pyrimidine bases thymine (T), cytosine (C), and uracil (U). Modified nucleotide bases can include other synthetic and natural nucleotide bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-porphyry uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Other useful nucleotide bases include those disclosed, for example, in U.S. Pat. No. 3,687,808.

Certain nucleotide base substitutions can be particularly useful for increasing the binding affinity of the antisense oligonucleotides of the invention. For example, 5-methylcytosine substitutions have been shown to increase nucleic acid duplex stability by 0.6 to 1.2° C. (Sanghvi et al., eds, Antisense Research and Applications, pp. 276-278, CRC Press, Boca Raton, Fla., 1993). Other useful nucleotide base substitutions include 5-substituted pyrimidines, 6-azapyrimidines and N-2, N-6 and O-6 substituted purines such as 2-aminopropyladenine, 5-propynyluracil and 5-propynylcytosine.

Antisense oligonucleotides of the invention also can be modified by chemical linkage to one or more moieties or conjugates that enhance the activity, cellular distribution or cellular uptake of the oligonucleotide. Such moieties include but are not limited to lipid moieties (e.g., a cholesterol moiety); cholic acid; a thioether moiety (e.g., hexyl-S-tritylthiol); a thiocholesterol moiety; an aliphatic chain (e.g., dodecandiol or undecyl residues); a phospholipid moiety (e.g., di-hexadecyl-rac-glycerol or triethyl-ammonium 1,2-di-O-hexadecyl-rac-glycero-3-H-phosphonate); a polyamine or a polyethylene glycol chain; adamantane acetic acid; a palmityl moiety; or an octadecylamine or hexylamino-carbonyl-oxycholesterol moiety. The preparation of such oligonucleotide conjugates is disclosed in, for example, U.S. Pat. Nos. 5,218,105 and 5,214,136.

It is not necessary for all nucleotide base positions in a given antisense oligonucleotide to be uniformly modified. More than one of the aforementioned modifications can be incorporated into a single oligonucleotide or even at a single nucleoside within an oligonucleotide. The present invention also includes antisense oligonucleotides that are chimeric oligonucleotides. “Chimeric” antisense oligonucleotides can contain two or more chemically distinct regions, each made up of at least one monomer unit (e.g., a nucleotide in the case of an oligonucleotide). Chimeric oligonucleotides typically contain at least one region wherein the oligonucleotide is modified so as to confer, for example, increased resistance to nuclease degradation, increased cellular uptake, and/or increased affinity for the target nucleic acid. For example, a region of a chimeric oligonucleotide can serve as a substrate for an enzyme such as RNase H, which is capable of cleaving the RNA strand of an RNA:DNA duplex such as that formed between a target mRNA and an antisense oligonucleotide. Cleavage of such a duplex by RNase H, therefore, can greatly enhance the effectiveness of an antisense oligonucleotide.

Antisense molecules in accordance with the invention can include enzymatic ribonucleic acid molecules that can cleave other ribonucleic acid molecules (ribozymes). Antisense technologies involving ribozymes have shown great utility in research, diagnostic and therapeutic contexts. Methods for designing and using ribozymes are well known, and have been extensively described. Ribozymes in general are described, for example, in U.S. Pat. Nos. 5,254,678; 5,496,698; 5,525,468; and 5,616,459. U.S. Pat. Nos. 5,874,414 and 6,015,794 describe trans-splicing ribozymes. Hairpin ribozymes are described, for example, in U.S. Pat. Nos. 5,631,115; 5,631,359; 5,646,020; 5,837,855 and 6,022,962. U.S. Pat. No. 6,307,041 describes circular, hairpin, circular/hairpin, lariat, and hairpin-lariat hammerhead ribozymes. Ribozymes can include deoxyribonucleotides (see, e.g., U.S. Pat. Nos. 5,652,094; 6,096,715 and 6,140,491). Such ribozymes are often referred to as (nucleozymes). Ribozymes can include modified ribonucleotides. Base-modified enzymatic nucleic acids are described, for example, in U.S. Pat. Nos. 5,672,511; 5,767,263; 5,879,938 and 5,891,684. U.S. Pat. No. 6,204,027 describes ribozymes having 2′-O substituted nucleotides in the flanking sequences. U.S. Pat. No. 5,545,729 describes stabilized ribozyme analogs. Other ribozymes having specialized properties have been described, for example, in U.S. Pat. No. 5,942,395 (describing chimeric ribozymes that include a snoRNA stabilizing motif), U.S. Pat. Nos. 6,265,167 and 5,908,779 (describing nuclear ribozymes), U.S. Pat. No. 5,994,124 (describing ribozyme-snRNA chimeric molecules having a catalytic activity for nuclear RNAs); and U.S. Pat. No. 5,650,502 (describing ribozyme analogs with rigid non-nucleotidic linkers).

The P2X2 antisense oligonucleotides of the invention are synthesized in vitro and do not include antisense compositions of biological origin, except for oligonucleotides that comprise the subject antisense oligonucleotides and have been purified from or isolated from biological material. Antisense oligonucleotides used in accordance with this invention can be conveniently produced through the well-known technique of solid phase synthesis. Equipment for such synthesis is commercially available from several vendors including, for example, Applied Biosystems (Foster City, Calif.). Any other means for such synthesis known in the art additionally or alternatively can be employed. Similar techniques also can be used to prepare modified oligonucleotides such as phosphorothioates or alkylated derivatives.

### Antisense Preparations and Methods for Use

The antisense oligonucleotides of the invention are useful for research (e.g., in developing assays to identify small molecule therapeutics), diagnostics, and for therapeutic use. For example, assays based on hybridization of antisense oligonucleotides to nucleic acids encoding P2X2 can be used to evaluate levels of P2X2 in a tissue sample. Hybridization of the antisense oligonucleotides of the invention with a nucleic acid encoding P2X2 can be detected by means known in the art. Such means can include conjugation of an enzyme to the antisense oligonucleotide, radiolabeling of the antisense oligonucleotide, or any other suitable means of detection.

Those of skill in the art can harness the specificity and sensitivity of antisense technology for therapeutic use. Antisense oligonucleotides have been employed as therapeutic moieties in the treatment of disease states in animals, including humans. For therapeutic methods, the cells or tissues are typically within a vertebrate (e.g., a mammal such as a human).

The invention provides therapeutic methods for treating conditions involving abnormal expression (e.g., over-production) or altered function of the P2X2 purinoreceptor. By these methods, antisense oligonucleotides in accordance with the invention are administered to a subject (e.g., a human) suspected of having a disease or condition (e.g., chronic pain or irritable bowel syndrome) that can be alleviated by modulating the expression of P2X2. Typically, one or more antisense oligonucleotides can be administered to a subject suspected of having a disease or condition associated with the expression of P2X2. The antisense oligonucleotide can be in a pharmaceutically acceptable carrier or diluent, and can be administered in amounts and for periods of time that will vary depending upon the nature of the particular disease, its severity, and the subject's overall condition. Typically, the antisense oligonucleotide is administered in an inhibitory amount (i.e., in an amount that is effective for inhibiting the production of P2X2 in the cells or tissues contacted by the antisense oligonucleotides). The antisense oligonucleotides and methods of the invention also can be used prophylactically, e.g., to minimize pain in a subject that exhibits abnormal expression of P2X2 or altered P2X2 function.

The ability of a P2X2 antisense oligonucleotide to inhibit expression and/or production of P2X2 can be assessed, for example, by measuring levels of P2X2 mRNA or protein in a subject before and after treatment. Methods for measuring mRNA and protein levels in tissues or biological samples are known in the art. If the subject is a research animal, for example, P2X2 levels in the brain can be assessed by in situ hybridization or immunostaining following euthanasia. Indirect methods can be used to evaluate the effectiveness of P2X2 antisense oligonucleotides in live subjects. For example, reduced expression of P2X2 can be inferred from reduced sensitivity to painful stimuli. As described in the Examples below, animal models can be used to study the development, maintenance, and relief of chronic neuropathic or inflammatory pain. Animals subjected to these models generally develop thermal hyperalgesia (i.e., an increased response to a stimulus that is normally painful) and/or allodynia (i.e., pain due to a stimulus that is not normally painful). Sensitivity to mechanical and thermal stimuli can be assessed (see Bennett, Methods in Pain Research, pp. 67-91, Kruger, Ed., 2001) to evaluate the effectiveness of P2X2 antisense treatment.

Methods for formulating and subsequently administering therapeutic compositions are well known to those skilled in the art. Dosing is generally dependent on the severity and responsiveness of the disease state to be treated, with the course of treatment lasting from several days to several months, or until a cure is effected or a diminution of the disease state is achieved. Persons of ordinary skill in the art routinely determine optimum dosages, dosing methodologies and repetition rates. Optimum dosages can vary depending on the relative potency of individual oligonucleotides, and can generally be estimated based on EC₅₀ values found to be effective in in vitro and in vivo animal models. Typically, dosage is from 0.01 μg to 100 g per kg of body weight, and may be given once or more daily, weekly, or even less often. Dosage and dosing schedules vary depending on route of administration (e.g., systemic doses typically are greater than intrathecal or epidural doses). Following successful treatment, it may be desirable to have the patient undergo maintenance therapy to prevent the recurrence of the disease state.

The present invention provides pharmaceutical compositions and formulations that include the P2X2 antisense oligonucleotides of the invention. P2X2 antisense oligonucleotides therefore can be admixed, encapsulated, conjugated or otherwise associated with other molecules, molecular structures, or mixtures of oligonucleotides such as, for example, liposomes, receptor targeted molecules, or oral, rectal, topical or other formulations, for assisting in uptake, distribution and/or absorption.

A “pharmaceutically acceptable carrier” (also referred to herein as an “excipient”) is a pharmaceutically acceptable solvent, suspending agent, or any other pharmacologically inert vehicle for delivering one or more therapeutic molecules (e.g., P2X2 antisense oligonucleotides) to a subject. Pharmaceutically acceptable carriers can be liquid or solid, and can be selected with the planned manner of administration in mind so as to provide for the desired bulk, consistency, and other pertinent transport and chemical properties, when combined with one or more of therapeutic molecules and any other components of a given pharmaceutical composition. Typical pharmaceutically acceptable carriers that do not deleteriously react with nucleic acids include, by way of example and not limitation: water; saline solution; binding agents (e.g., polyvinylpyrrolidone or hydroxypropyl methylcellulose); fillers (e.g., lactose and other sugars, gelatin, or calcium sulfate); lubricants (e.g., starch, polyethylene glycol, or sodium acetate); disintegrates (e.g., starch or sodium starch glycolate); and wetting agents (e.g., sodium lauryl sulfate).

The pharmaceutical compositions of the present invention can be administered by a number of methods depending upon whether local or systemic treatment is desired and depending upon the area to be treated. Administration can be, for example, topical (e.g., transdermal, ophthalmic, or intranasal); pulmonary (e.g., by inhalation or insufflation of powders or aerosols); oral; or parenteral (e.g., by subcutaneous, intrathecal, intraventricular, intramuscular, or intraperitoneal injection, or by intravenous drip). Administration can be rapid (e.g., by injection) or can occur over a period of time (e.g., by slow infusion or administration of slow release formulations). For treating tissues in the central nervous system, antisense oligonucleotides can be administered by injection or infusion into the cerebrospinal fluid, preferably with one or more agents capable of promoting penetration of the antisense oligonucleotide across the blood-brain barrier.

Formulations for topical administration of antisense oligonucleotides include, for example, sterile and non-sterile aqueous solutions, non-aqueous solutions in common solvents such as alcohols, or solutions in liquid or solid oil bases. Such solutions also can contain buffers, diluents and other suitable additives. Pharmaceutical compositions and formulations for topical administration can include transdermal patches, ointments, lotions, creams, gels, drops, suppositories, sprays, liquids, and powders. Coated condoms, gloves and the like also may be useful. Conventional pharmaceutical carriers, aqueous, powder or oily bases, thickeners and the like may be necessary or desirable.

Compositions and formulations for oral administration include, for example, powders or granules, suspensions or solutions in water or non-aqueous media, capsules, sachets, or tablets. Such compositions also can incorporate thickeners, flavoring agents, diluents, emulsifiers, dispersing aids, or binders. Oligonucleotides with at least one 2′-O-methoxyethyl modification (described above) may be particularly useful for oral administration.

Compositions and formulations for parenteral, intrathecal or intraventricular administration can include sterile aqueous solutions, which also can contain buffers, diluents and other suitable additives (e.g., penetration enhancers, carrier molecules and other pharmaceutically acceptable carriers).

Pharmaceutical compositions of the present invention include, but are not limited to, solutions, emulsions, aqueous suspensions, and liposome-containing formulations. These compositions can be generated from a variety of components that include, for example, preformed liquids, self-emulsifying solids and self-emulsifying semisolids. Emulsions are often biphasic systems comprising of two immiscible liquid phases intimately mixed and dispersed with each other; in general, emulsions are either of the water-in-oil (w/o) or oil-in-water (o/w) variety. Emulsion formulations have been widely used for oral delivery of therapeutics due to their ease of formulation and efficacy of solubilization, absorption, and bioavailability.

Liposomes are vesicles that have a membrane formed from a lipophilic material and an aqueous interior that can contain the antisense composition to be delivered. Liposomes can be particularly useful due to their specificity and the duration of action they offer from the standpoint of drug delivery. Liposome compositions can be formed, for example, from phosphatidylcholine, dimyristoyl phosphatidylcholine, dipalmitoyl phosphatidylcholine, dimyristoyl phosphatidylglycerol, or dioleoyl phosphatidylethanolamine. Numerous lipophilic agents are commercially available, including Lipofectin® (Invitrogen/Life Technologies, Carlsbad, Calif.) and Effectene™ (Qiagen, Valencia, Calif.).

The P2X2 antisense oligonucleotides of the invention further encompass any pharmaceutically acceptable salts, esters, or salts of such esters, or any other molecule which, upon administration to an animal including a human, is capable of providing (directly or indirectly) the biologically active metabolite or residue thereof. Accordingly, for example, the invention provides pharmaceutically acceptable salts of P2X2 antisense oligonucleotides, prodrugs and pharmaceutically acceptable salts of such prodrugs, and other bioequivalents. The term “prodrug” indicates a therapeutic agent that is prepared in an inactive form and is converted to an active form (i.e., drug) within the body or cells thereof by the action of endogenous enzymes or other chemicals and/or conditions. The term “pharmaceutically acceptable salts” refers to physiologically and pharmaceutically acceptable salts of the oligonucleotides of the invention (i.e., salts that retain the desired biological activity of the parent oligonucleotide without imparting undesired toxicological effects). Examples of pharmaceutically acceptable salts of oligonucleotides include, but are not limited to, salts formed with cations (e.g., sodium, potassium, calcium, or polyamines such as spermine); acid addition salts formed with inorganic acids (e.g., hydrochloric acid, hydrobromic acid, sulfuric acid, phosphoric acid, or nitric acid); salts formed with organic acids (e.g., acetic acid, citric acid, oxalic acid, palmitic acid, or fumaric acid); and salts formed from elemental anions (e.g., chlorine, bromine, and iodine).

Pharmaceutical compositions containing the antisense oligonucleotides of the present invention also can incorporate penetration enhancers that promote the efficient delivery of nucleic acids, particularly oligonucleotides, to the skin of animals. Penetration enhancers can enhance the diffusion of both lipophilic and non-lipophilic drugs across cell membranes. Penetration enhancers can be classified as belonging to one of five broad categories, i.e., surfactants (e.g., sodium lauryl sulfate, polyoxyethylene-9-lauryl ether and polyoxyethylene-20-cetyl ether); fatty acids (e.g., oleic acid, lauric acid, myristic acid, palmitic acid, and stearic acid); bile salts (e.g., cholic acid, dehydrocholic acid, and deoxycholic acid); chelating agents (e.g., disodium ethylenediaminetetraacetate, citric acid, and salicylates); and non-chelating non-surfactants (e.g., unsaturated cyclic ureas).

Certain embodiments of the invention provide pharmaceutical compositions containing (a) one or more antisense oligonucleotides and (b) one or more other agents that function by a non-antisense mechanism. For example, anti-inflammatory drugs, including but not limited to nonsteroidal anti-inflammatory drugs and corticosteroids, and antiviral drugs, including but not limited to ribivirin, vidarabine, acyclovir and ganciclovir, can be included in compositions of the invention. Other non-antisense agents (e.g., chemotherapeutic agents) are also within the scope of this invention. Such combined molecules can be used together or sequentially.

The antisense compositions of the present invention additionally can contain other adjunct components conventionally found in pharmaceutical compositions. Thus, the compositions also can include compatible, pharmaceutically active materials such as, for example, antipruritics, astringents, local anesthetics or anti-inflammatory agents, or additional materials useful in physically formulating various dosage forms of the compositions of the present invention, such as dyes, flavoring agents, preservatives, antioxidants, opacifiers, thickening agents and stabilizers. Furthermore, the composition can be mixed with auxiliary agents, e.g., lubricants, preservatives, stabilizers, wetting agents, emulsifiers, salts for influencing osmotic pressure, buffers, colorings, flavorings, and aromatic substances. When added, however, such materials should not unduly interfere with the biological activities of the antisense components within the compositions of the present invention. The formulations can be sterilized and, if desired, and the like which do not deleteriously interact with the nucleic acid(s) of the formulation.

The pharmaceutical formulations of the present invention, which can be presented conveniently in unit dosage form, can be prepared according to conventional techniques well known in the pharmaceutical industry. Such techniques include the step of bringing into association the active ingredients (e.g., the P2X2 antisense oligonucleotides of the invention) with the desired pharmaceutical carrier(s) or excipient(s). Typically, the formulations can be prepared by uniformly and bringing the active ingredients into intimate association with liquid carriers or finely divided solid carriers or both, and then, if necessary, shaping the product. Formulations can be sterilized if desired, provided that the method of sterilization does not interfere with the effectiveness of the antisense oligonucleotide contained in the formulation.

The compositions of the present invention can be formulated into any of many possible dosage forms such as, but not limited to, tablets, capsules, liquid syrups, soft gels, suppositories, and enemas. The compositions of the present invention also can be formulated as suspensions in aqueous, non-aqueous or mixed media. Aqueous suspensions further can contain substances that increase the viscosity of the suspension including, for example, sodium carboxymethylcellulose, sorbitol, and/or dextran. Suspensions also can contain stabilizers.

### Nucleic Acid Constructs

Nucleic acid constructs (e.g., a plasmid vector) are capable of transporting a nucleic acid into a host cell. Suitable host cells include prokaryotic or eukaryotic cells (e.g., bacterial cells such as E. coli, insect cells, yeast cells, and mammalian cells). Some constructs are capable of autonomously replicating in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell and are replicated with the host genome.

Nucleic acid constructs can be, for example, plasmid vectors or viral vectors (e.g., replication defective retroviruses, adenoviruses, and adeno-associated viruses). Nucleic acid constructs include one or more regulatory sequences operably linked to the nucleic acid of interest (e.g., a nucleic acid encoding a transcript that specifically hybridizes to a P2X2 mRNA in its native form). With respect to regulatory elements, “operably linked” means that the regulatory sequence and the nucleic acid of interest are positioned such that nucleotide sequence is transcribed (e.g., when the vector is introduced into the host cell).

Regulatory sequences include promoters, enhancers and other expression control elements (e.g., polyadenylation signals). (See, e.g., Goeddel, Gene Expression Technology: Methods in Enzymology, 185, Academic Press, San Diego, Calif., 1990). Regulatory sequences include those that direct constitutive expression of a nucleotide sequence in many types of host cells and that direct expression of the nucleotide sequence only in certain host cells (e.g., cell type or tissue-specific regulatory sequences).

### Articles of Manufacture

Antisense oligonucleotides of the invention can be combined with packaging material and sold as kits for reducing P2X2 expression. Components and methods for producing articles of manufacture such as kits are well known. An article of manufacture may combine one or more of the antisense oligonucleotides set out in the above sections. In addition, the article of manufacture further may include buffers, hybridization reagents, or other control reagents for reducing or monitoring reduced P2X2 expression. Instructions describing how the antisense oligonucleotides are effective for reducing P2X2 expression can be included in such kits.

The invention will be further described in the following examples, which do not limit the scope of the invention described in the claims.

### EXAMPLES

### Example 1

### Materials and Methods

### Determination of Accessible Sites Within the P2X2 mRNA and Design of P2X2 Antisense Oligonucleotides

Accessible regions of rat P2X2 mRNA (as determined by the RiboTAG™ method) are shown in Table 1.

TABLE 1
Accessible sequences within rat P2X2 mRNA

| Start | End |
|---|---|
| 231 | 249 |
| 589 | 617 |
| 650 | 668 |
| 829 | 846 |
| 940 | 957 |
| 1246 | 1273 |
| 1429 | 1446 |

### Methods for Evaluating Pain in Rats Treated with Antisense P2X2

Two different models of chronic pain were used to evaluate the effects of P2X2 knock-down by intrathecally administered antisense oligonucleotides. Both models included the following six steps (described in greater detail below):

(1) spinal catheterization;

(2) nociceptive testing (baseline);

(3) induction of chronic neuropathic or inflammatory pain;

(4) nociceptive testing (post-injury);

(5) antisense injection; and

(6) nociceptive testing (post-treatment).

### Spinal Catheterization:

Male Sprague Dawley rats weighing between 200 and 250 g were obtained from Harlan (Indianapolis, Ind.). Rats were deeply anesthetized with a mixture containing 75 mg/kg ketamine, 5 mg/kg xylazine, and 1 mg/kg acepromazine, and a catheter (8.5 cm; PE-10) was passed to the lumbosacral intrathecal space through an incision in the dura over the atlantooccipital joint. Following surgery, animals were kept on a warming blanket and were periodically turned and carefully observed until completely recovered from anesthesia. Animals were allowed to recover for at least 3 days before being subjected to models of chronic pain.

### Mechanical Nociceptive Testing:

Baseline, post-injury, and post-treatment values for mechanical sensitivity were evaluated with calibrated monofilaments (von Frey filaments) according to the up-down method (Chaplan et al., 1994, J. Neurosci. Methods, 53:55-63). Animals were placed on a wire mesh platform and allowed to acclimate to their surroundings for a minimum of 10 minutes before testing. Filaments of increasing force were sequentially applied to the plantar surface of the paw just to the point of bending, and held for three seconds. The behavioral endpoint of the stimulus (achieved when the stimulus was of sufficient force) was the point at which the animal would lick, withdraw and/or shake the paw. The force or pressure required to cause a paw withdrawal was recorded as a measure of threshold to noxious mechanical stimuli for each hind-paw. The mean and standard error of the mean (SEM) were determined for each animal in each treatment group. The data were analyzed using repeated measures ANOVA followed by the Bonferonni post-hoc test. Since this stimulus is normally not considered painful and rats do not normally respond to filaments in the range selected, significant injury-induced increases in responsiveness in this test were interpreted as a measure of mechanical allodynia.

### Thermal Nociceptive Testing:

Baseline, post-injury, and post-treatment thermal sensitivities were determined by measuring withdrawal latencies in response to radiant heat stimuli delivered to the plantar surface of the hind-paws (Hargreaves et al., 1988, Pain, 32:77-88). Animals were placed on a plexiglass platform and allowed to acclimate for a minimum of 10 minutes. A radiant heat source was directed to the plantar surface, and the time to withdrawal was measured. For each paw, the withdrawal latency was determined by averaging three measurements separated by at least 5 minutes. The heating device was set to automatically shut off after a programmed period of time to avoid damage to the skin of unresponsive animals. The data were analyzed using repeated measures ANOVA followed by the Bonferonni post-hoc test. Significant injury-induced increases in thermal response latencies were considered to be a measure of thermal hyperalgesia since the stimulus is normally in the noxious range.

### Induction of Chronic Neuropathic Pain:

The Spinal Nerve Ligation (SNL) model (Kim & Chung, 1992, Pain, 50:355-63) was used to induce chronic neuropathic pain. Rats were anesthetized with isoflurane, the L5 transverse process was removed, and the L5 and L6 spinal nerves were tightly ligated with 6-0 silk suture. The wound was then closed with internal sutures and external staples. Control animals received a sham surgery consisting of removing the transverse process and exposing the L5 spinal nerve without ligating. All operations were performed on the left side.

### Induction of Chronic Inflammation:

The complete Freund's adjuvant (CFA) model of chronic peripheral inflammation was utilized (see, for example, Hylden et al., 1989, Pain, 37:229-43). Rats under light anesthesia received an injection of CFA (75 μl) into the left hindpaw using a sterile 1.0 ml syringe. A separate population of control rats was subjected to unilateral injection of saline.

### Antisense Design and Injection:

Oligonucleotides were dissolved in dH₂O and delivered into the intrathecal space in a volume of 5 ul per injection as previously described (see, for example, Bilsky et al., 1996, Neurosci. Lett., 220:155-158; Bilsky et al., 1996, J. Pharmacol. Exp. Ther., 277:491-501; and Vanderah et al., 1994, Neuroreport, 5:2601-2605). Antisense oligonucleotides were administered twice daily for 3 to 4 days, beginning on the afternoon following post-injury (baseline) nociceptive testing. Antisense oligonucleotides included the sequence GTA GTG GAT GCT GTT CTT GAT G (SEQ ID NO:3), which specifically hybridize to nucleotide bases 594 through 615 of SEQ ID NO:1. A mismatch oligonucleotide having the sequence GTA GTT GAG GCT CTT GTT GAT G (SEQ ID NO:4) was used as a control in animals analyzed for mRNA expression and as vehicle controls for the behavior experiments.

### Example 2

### Antisense Knockdown of P2X2 in Rat Spinal Cord Supports a Role for P2X2 in Chronic Neuropathic and Inflammatory Pain

Antisense oligonucleotides were designed by the RiboTAG™ method and used to evaluate the role of P2X2 in chronic pain. Thermal (radiant heat) and mechanical (von Frey) pain thresholds were obtained before and after induction of chronic pain (neuropathic or inflammatory, as described in Example 1, above). Antisense oligonucleotides (45 μg) or vehicle controls were delivered twice daily for 3 to 4 days, and thermal and mechanical thresholds were reassessed.

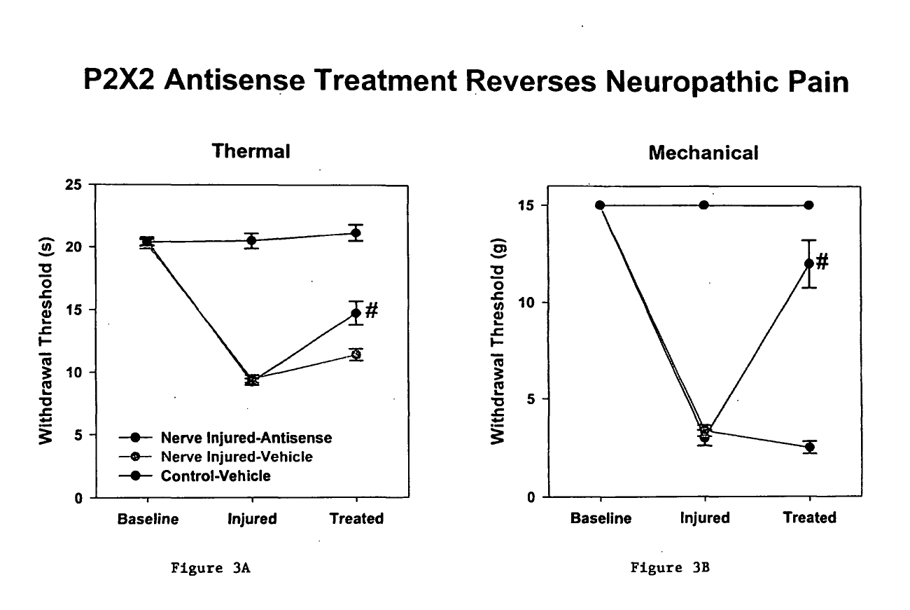

FIG. 3A shows the effect of P2X2 antisense oligonucleotides on mechanical thermal pain sensation. Normal rats responded to a noxious heat stimulus applied to their hindpaws with an average latency of 20 seconds (‘Baseline’). In animals in which a model of chronic nerve-injury induced (neuropathic) pain has been induced, the response latency decreased to around 10 seconds (‘Injured’). This drop is analogous to the abnormal pain sensitivity observed in human patients with chronic nerve-injury related pain such as in diabetic neuropathy. Following three days of P2X2 antisense treatment, there was a significant reduction in the nerve-injury induced hypersensitivity to thermal stimuli (‘Treated’).

FIG. 3B shows the effect of P2X2 antisense oligonucleotides on mechanical pain sensation. Normal animals rarely responded to stimuli of less than 15 g (‘Baseline’). In animals with nerve-injury, animals withdrew from stimuli of only a few grams (‘Injured’). Following three days of P2X2 antisense treatment, there was a significant reduction in the nerve-injury induced hypersensitivity to mechanical stimuli (‘Treated’).

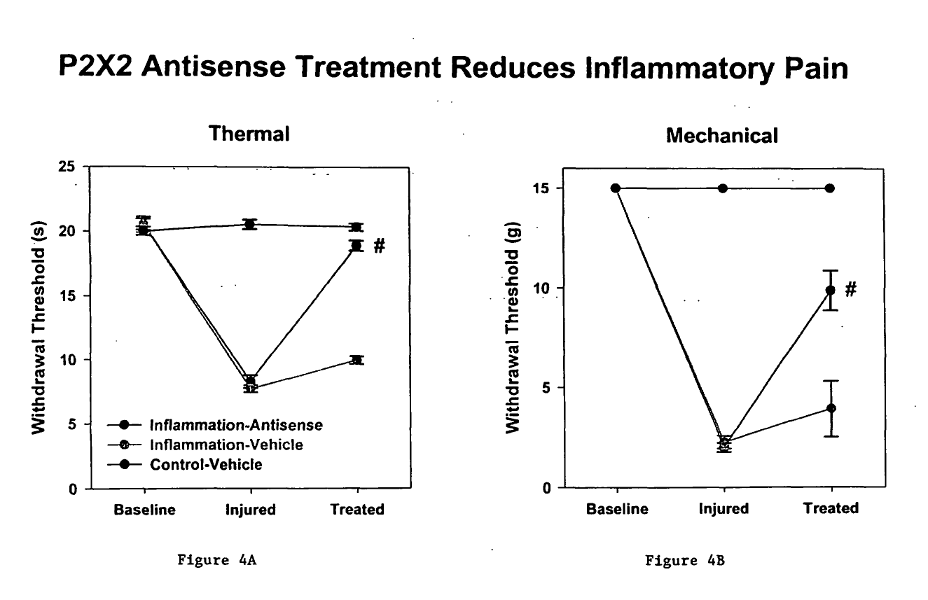

As shown in FIG. 4A and FIG. 4B, animals subjected to inflammation also were significantly more sensitive to thermal and mechanical stimuli (as evidenced by the decreases in their response thresholds compared to pre-inflammation baseline (‘Baseline’) and uninflamed controls). Following three days of treatment, there was a significant reduction in inflammatory-induced hypersensitivity to both thermal and mechanical stimuli (‘Treated’).

### Example 3

### Quantitative TaqMan RT-PCR Analysis of P2X2 After Antisense Treatment

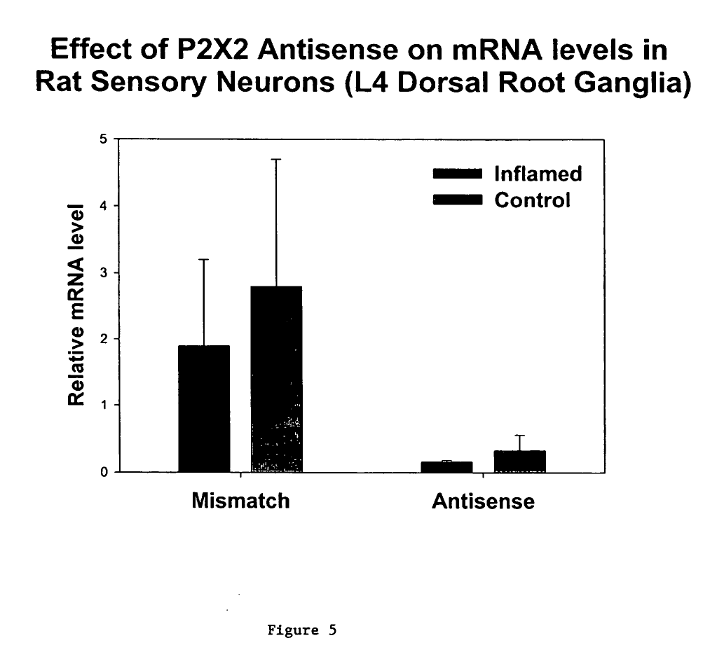

Quantitative PCR method was used to evaluate P2X2 mRNA levels in control animals, and in animals with a chronic inflammation in one of the hindpaws, treated with P2X2 antisense or a mismatch. Treatment with antisense reduced the level of P2X2-mRNA in both inflamed and control animals (FIG. 5).

TaqMan PCR was carried out using an ABI 7700 sequence detector (Perkin Elmer) on the cDNA samples. TaqMan primer and probe sets were designed from sequences in the GeneBank database using Primer Express (Perkin Elmer). The primers and probe used for the analysis were: ACC AGG ATC GAG GTT ACC CC (forward primer, SEQ ID NO:5), GAG CTG TGA ACC CTC ATG CTC (reverse primer, SEQ ID NO:6), and TCC CAG ACC TTG GGA ACA TGC CC (TaqMan probe, SEQ ID NO:7).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An isolated antisense oligonucleotide that specifically hybridizes within an accessible region of P2X2 mRNA in its native form, wherein said antisense oligonucleotide inhibits the production of P2X2.

2. An isolated antisense oligonucleotide consisting essentially of 10 to 50 nucleotides, wherein said oligonucleotide specifically hybridizes within an accessible region of P2X2 mRNA, said region defined by nucleotides 231 through 249, 589 through 617, 650 through 668, 829 through 846, 940 through 957, 1246 through 1273, or 1429 through 1446 of SEQ ID NO:1, and wherein said oligonucleotide inhibits the production of P2X2.

3. A composition comprising the isolated antisense oligonucleotide of claim 2.

4. The composition of claim 3, wherein said composition comprises a plurality of isolated antisense oligonucleotides, wherein each antisense oligonucleotide specifically hybridizes to a different accessible region.

5. A nucleic acid construct comprising a regulatory element operably linked to a nucleic acid encoding a transcript, wherein said transcript specifically hybridizes within one or more accessible regions of P2X2 mRNA in its native form.

6. A host cell comprising the nucleic acid construct of claim 5.

7. A method of decreasing production of P2X2 in cells or tissues, comprising contacting said cells or tissues with an antisense oligonucleotide that specifically hybridizes within an accessible region of P2X2.

8. A method for modulating pain in a mammal, said method comprising administering the isolated antisense oligonucleotide of claim 1 to said mammal.
